(19) United States
(12) Patent Application Publication
(10) Pub. No.: US 2017/0046120 A1
(43) Pub. Date: Feb. 16, 2017

(54) Customizable Personal Sound Delivery System

(71) Applicant: Audeara Pty Ltd., St. Lucia (AU)

(72) Inventors: Christopher Arnold Jeffery, St. Lucia (AU); James Alexander Fielding, St. Lucia (AU); Logan Stuart Gardner, St. Lucia (AU); Alexander John Afflick, St. Lucia (AU); Alexander Gilmour, St. Lucia (AU)

(21) Appl. No.: 15/196,256

(22) Filed: Jun. 29, 2016

(30) Foreign Application Priority Data
Jun. 29, 2015 (AU) AU2015902532

(51) Int. Cl. G06F 3/16; A61B 5/12; A61B 5/00; H04R 3/04

(52) U.S. Cl. CPC G06F 3/165; H04R 3/04; A61B 5/123; A61B 5/6898; A61B 5/7282; A61B 5/7475

(57) ABSTRACT

A sound delivery system includes a processing assembly with a user interface coupled to the at least one processing assembly. At least one audio transducer is provided for delivering sound to a user. The audio transducer is responsive to the processing assembly. Typically the audio transducer is a loudspeaker of a pair of headphones or earbuds, though it may also be a bone conduction transducer. The at least one processing assembly is arranged to determine compensatory weights at each of a number of audio frequencies for the user on the basis of user responses via the interface to sounds delivered via the audio transducer and to deliver audio signals to the user modified in accordance with the determined weights via the audio transducer.

### TECHNICAL FIELD

The present invention relates to sound delivery systems which involve audio transducers such as headphones, ear plugs or in some circumstances bone conduction transducers and which can be customized by a user to take into account the user's auditory response.

### BACKGROUND

Any references to methods, apparatus or documents of the prior art are not to be taken as constituting any evidence or admission that they formed, or form part of the common general knowledge.

Different people have different auditory responses. For example, younger people are typically able to hear audio frequencies at higher frequencies than older people. As people age, or as a result of hearing damage due to exposure to loud sounds, their hearing tends to deteriorate and their auditory response across a range of frequencies changes.

It is known to provide programmable hearing aids that can, through testing of the user by an audiologist, be set to compensate for the user's deteriorated hearing loss. However, such systems require that the user makes an appointment to have a hearing aid test and that the hearing aid be set by the technician.

It is an object of the present invention to provide a customizable personal sound delivery system that a user can conveniently use and which can automatically test the user and make adjustments to its sound delivery parameters on the basis of the test results.

### SUMMARY OF THE INVENTION

According to a first aspect of the present invention there is provided a sound delivery system including: at least one processing assembly; an interface coupled to the at least one processing assembly; and at least one audio transducer responsive to the at least one processing assembly for delivering sound to a user; wherein the at least one processing assembly is arranged to determine compensatory weights at each of a number of audio frequencies for the user on the basis of user responses via the interface to sounds delivered via the audio transducer and to deliver audio signals to the user modified in accordance with the determined weights via said audio transducer.

In a preferred embodiment of the invention the sound delivery system comprises an interface portion which includes the user interface and a transducer portion which includes the at least one audio transducer, wherein the first interface portion and the transducer portion include corresponding data communication assemblies for data communication there between.

Preferably the at least one processing assembly includes: at least one interface processor that is mounted within the interface portion and coupled to the user interface; and at least one transducer processor that is mounted within the transducer portion and arranged to process sound signals for delivery as sound by said audio transducer.

Preferably the data communication assemblies are arranged for wireless data communication. For example the data communication assemblies may be arranged to implement data communication according to the Bluetooth standard.

In a preferred embodiment of the invention the interface portion comprises a smartphone though it could alternatively be a tablet, laptop or desktop computer, for example.

According to another aspect of the present invention there is provided a sound delivery system including: at least one processing assembly; an interface coupled to the at least one processing assembly; at least one audio transducer responsive to the at least one processing assembly for delivering sound to a user; and an electronic memory accessible by the at least one processing assembly storing: instructions for the processor to determine compensatory weights at each of a number of audio frequencies for the user on the basis of user responses via the interface to sounds delivered via the audio transducer and to deliver audio signals to the user modified in accordance with the determined weights via said audio transducer.

An automatic audiological testing apparatus including: at least one processor; an electronic memory in communication with the processor and containing instructions for execution thereof; and a user interface in communication with the processor; wherein the electronic memory stores instructions for the processor to determine compensatory weights at each of a number of audio frequencies for the user on the basis of user responses via the interface to sounds at a number of different frequencies.

According to a further aspect of the present invention there is provided a set of headphones including right and left loudspeakers for delivery of sounds to a user: at least one processor configured to receive gain adjustment weights for the user for each of a number of predetermined frequencies; wherein the processor is arranged to convert an audio signal into the frequency domain, apply the gain adjustment weights to the audio signal in the frequency domain and convert the adjusted audio signal back into the time domain for delivery of an adjusted audio signal to the user via the loudspeakers.

According to another aspect of the present invention there is provided a method for sound delivery to a user including: presenting sounds of different frequencies and prompts to a user in order to determine an audiological model of the user comprising a set of gain adjustment weights for each of the different frequencies; and adjusting audio signals according to the adjustment weights to thereby deliver adjusted audio signals to the user to compensate for hearing deficiencies of the user.

Preferably the method includes facilitating adjustment of the weights by the user to introduce frequency equalization parameters selected by the user for each of a number of frequency bands.

It will therefore be realized that in one embodiment of the invention there is provided a sound delivery system that includes a processing assembly with a user interface coupled thereto. At least one audio transducer is provided for delivering sound to a user, which is responsive to the processing assembly. Typically the audio transducer is a loudspeaker of a pair of headphones or earbuds, though it may also be a bone conduction transducer. The at least one processing assembly is arranged to determine compensatory weights at each of a number of audio frequencies for the user on the basis of user responses via the interface to sounds delivered via the audio transducer and to deliver audio signals to the user modified in accordance with the determined weights via the audio transducer.

Additional features and advantages of the present invention are described in, and will be apparent from, the detailed description of the presently preferred embodiments and from the drawings.

### BRIEF DESCRIPTION OF THE DRAWINGS

Preferred features, embodiments and variations of the invention may be discerned from the following Detailed Description which provides sufficient information for those skilled in the art to perform the invention. The Detailed Description is not to be regarded as limiting the scope of the preceding Summary of the Invention in any way. The Detailed Description will make reference to a number of drawings as follows:

FIG. 1 is a high level diagram of a sound delivery system according to a preferred embodiment of the present invention in use;

FIG. 2 is a block diagram of electronic circuitry of a transducer portion of the sound delivery system;

FIGS. 3A-3D are a first portion of a circuit schematic generally corresponding to the block diagram of FIG. 2;

FIGS. 4A-4B are a second portion of the circuit schematic of FIGS. 3A-3D;

FIG. 5 is a high level block diagram of a user interface portion, in the form of a smartphone, of the sound delivery system.

FIGS. 6 to 10 are screen shots of screens presented to a user by the smartphone;

FIG. 11 is a block diagram illustrating a modeling method in accordance with an embodiment of the present invention;

FIGS. 12 and 13 are screen shots of screens presented to a user by the smartphone;

FIG. 14 comprises three frequency domain spectrograms. At left is the hearing response spectrogram of a person with normal hearing to a test audio signal. In the middle there is the hearing response spectrogram of a person with deteriorated hearing response in high frequency bands to the test audio signal. At right is the perceived audio response to the test signal subsequent to the test signal being gain adjusted to compensate for the high frequency loss; and,

FIG. 15 is a flowchart of the steps performed by the sound delivery system delivery of audio to a user.

### DETAILED DESCRIPTION

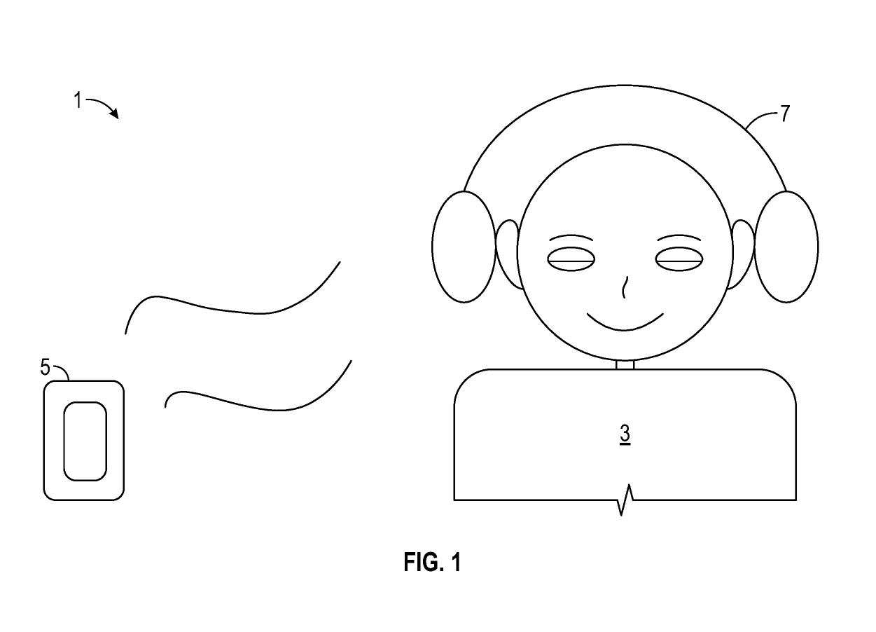

Referring now to FIG. 1, there is depicted a sound delivery system 1 in use and being applied to a user 3. In the presently described preferred embodiment the sound delivery system 1 is comprised of two major portions. A first portion comprises a smartphone 5, or other computational device such as a laptop, desktop or tablet computer. The smartphone 5 is in data communication with a second portion of the sound delivery system being a transducer portion, which in the present embodiment comprises headphones 7 though it might equally be a set of earbuds or some other sound delivery apparatus. The data communication between the smartphone 5 and the headphones 7 is by Bluetooth wireless in the presently described embodiment though of course it could be established otherwise, for example through a wired connection or by other wireless protocols.

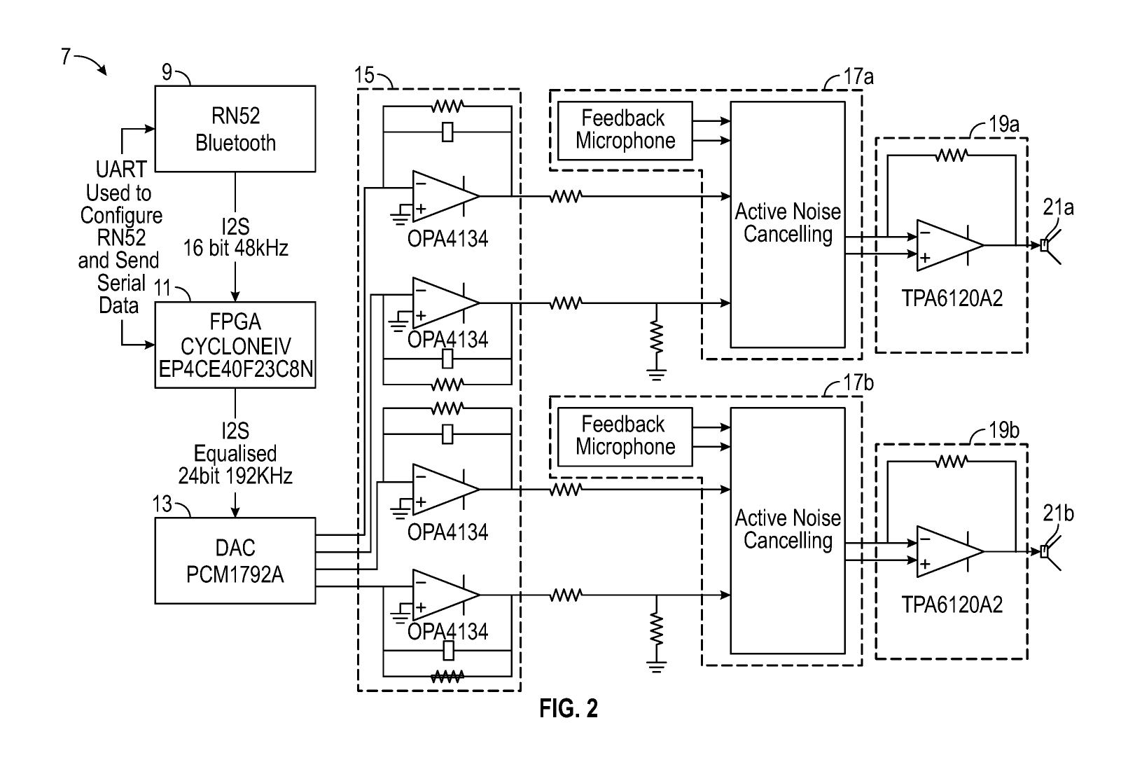

FIG. 2 is a high level block diagram of the electronic circuitry that is contained within the headphones 7. The circuitry includes a communications port 9 in the form of a Bluetooth port for communicating with the smartphone 5. A processor in the form of a field programmable gate array 11 is coupled to the Bluetooth port 8. As will be explained, the FPGA 11 is configured by uploading data from the smartphone 5 to apply “weights”, i.e. gain adjustment parameters, for different frequencies to an audio signal that it receives from smartphone. An output side of the FPGA 11 is coupled to a digital to analogue converter (DAC) 13. The DAC converts the digital audio signal from the FPGA into right and left stereo analogue signals which are applied via pre-amplifiers 15a, 15b, through noise cancelling modules 17a, 17b to output amplifiers 19a, 19b. The output amplifiers 19a, 19b drive electric signal to vibration transducers 21a, 21b. The transducers 21a, 21b are typically loudspeakers though they could alternatively be bone conduction transducers.

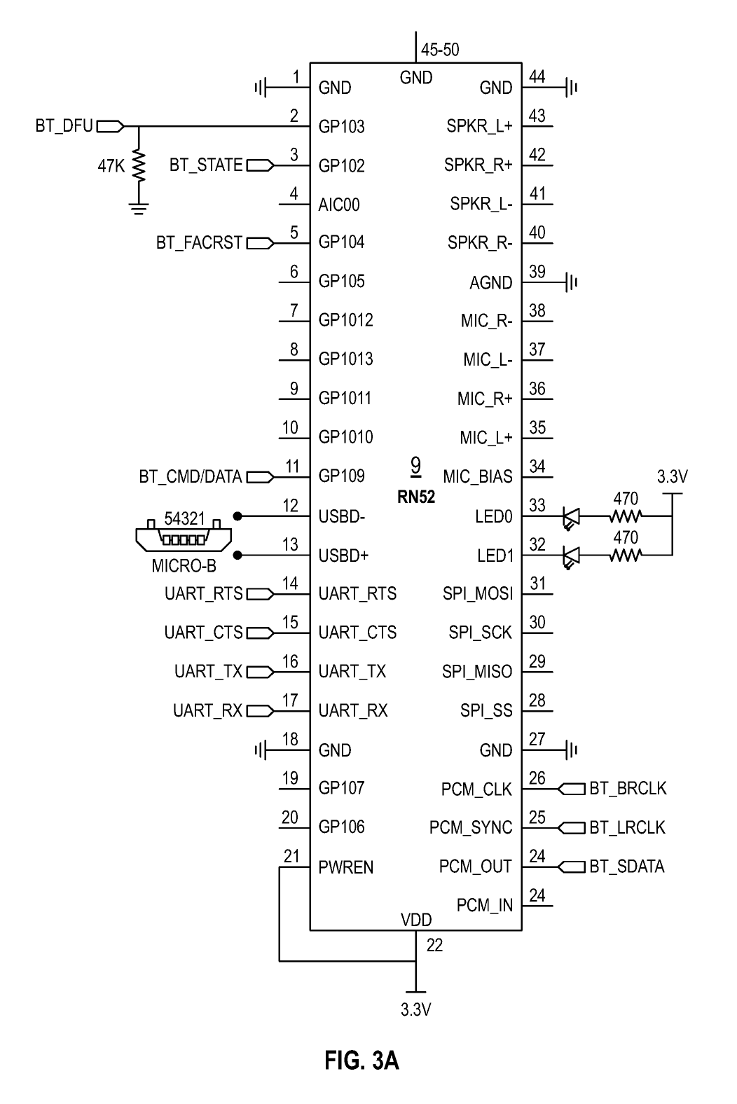

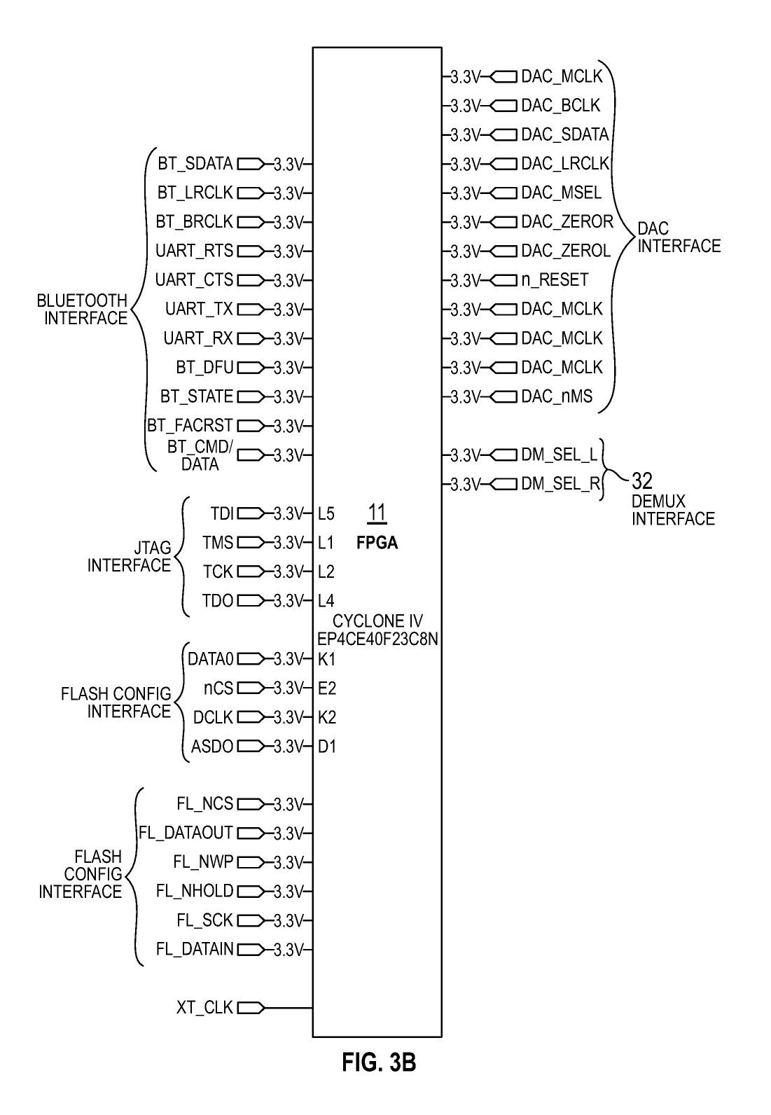

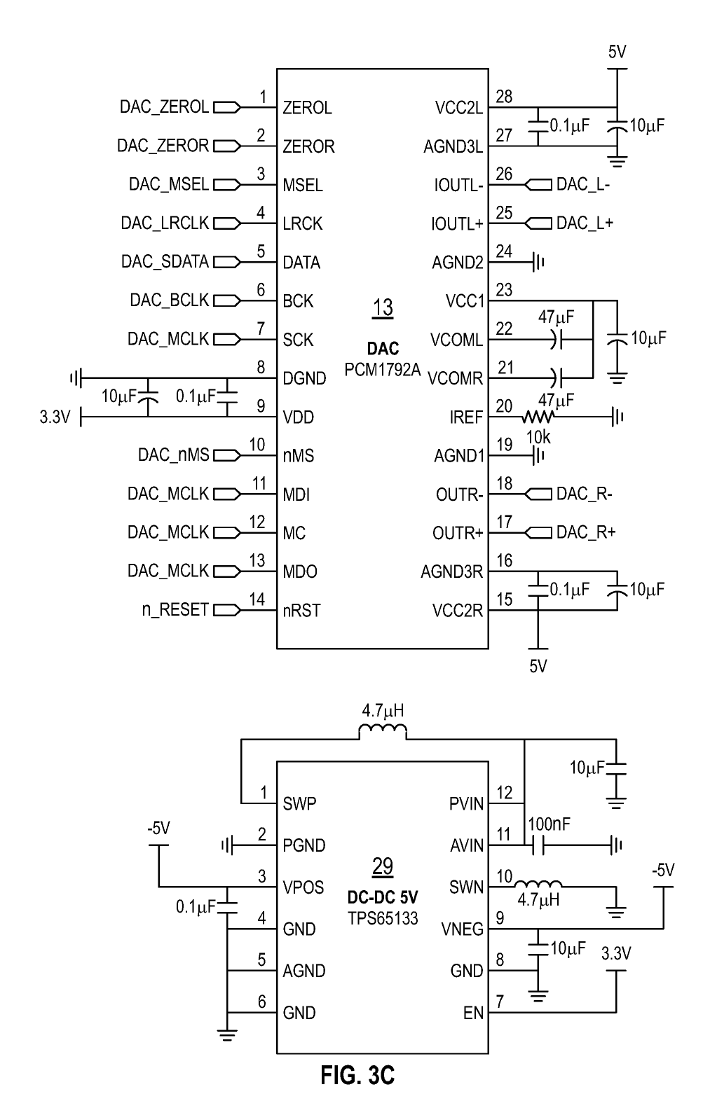

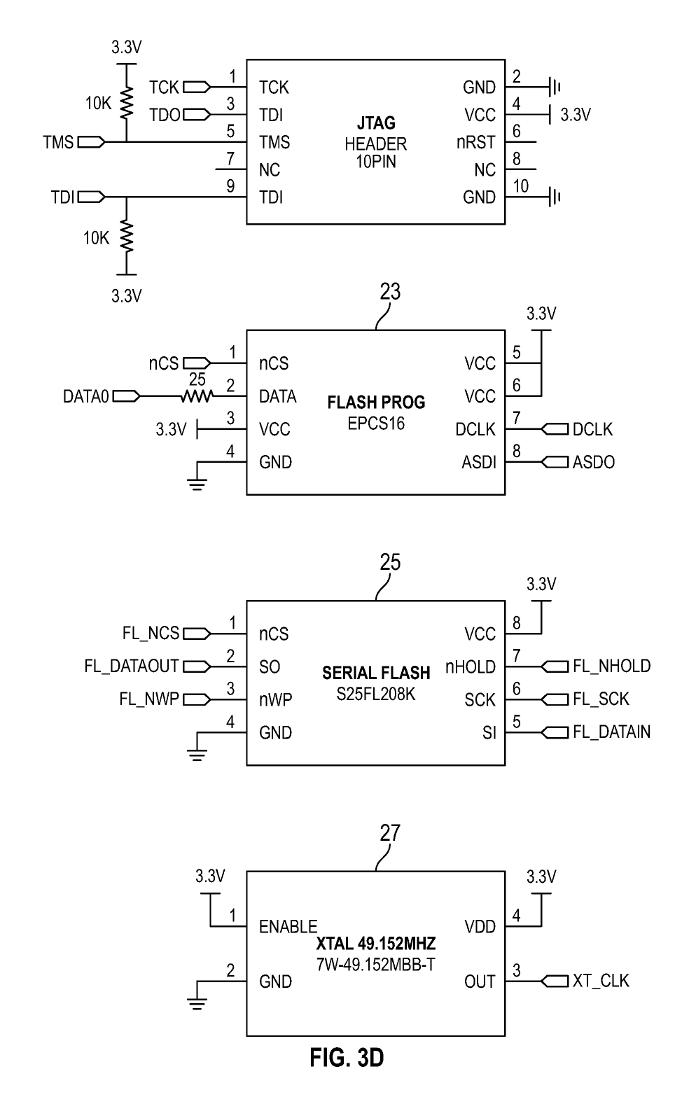

FIGS. 3A-3D are a first part of a circuit schematic corresponding to block diagram 3 and showing the Bluetooth port 9, FPGA 11 and DAC 13. FIGS. 3A-3D also shows programmable flash components 23 and 25 which are used to configure the FPGA, for example to set the frequency gain adjustments weights that the FPGA will apply to an audio signal in use. The FPGA 11 is a Cyclone IV EP4CE40F integrated circuit that is manufactured by Altera Corporation and which is configured to perform Fast Fourier Transforms on audio signals received via the Bluetooth port, apply the gain weights in the frequency domain and then perform an Inverse Fast Fourier Transform to convert the digital signal back to the time domain.

FIGS. 3A-3D also shows a clock module 27 and a power supply chip 29 for applying power to the various components. All of these components are readily available commercially.

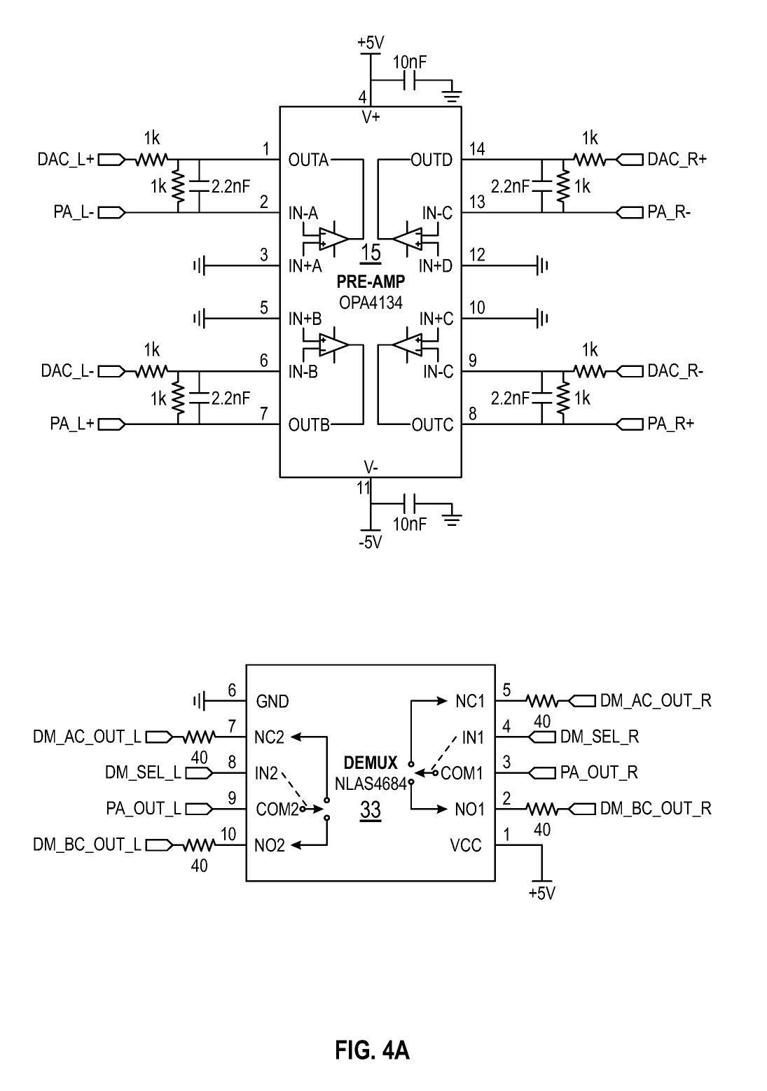

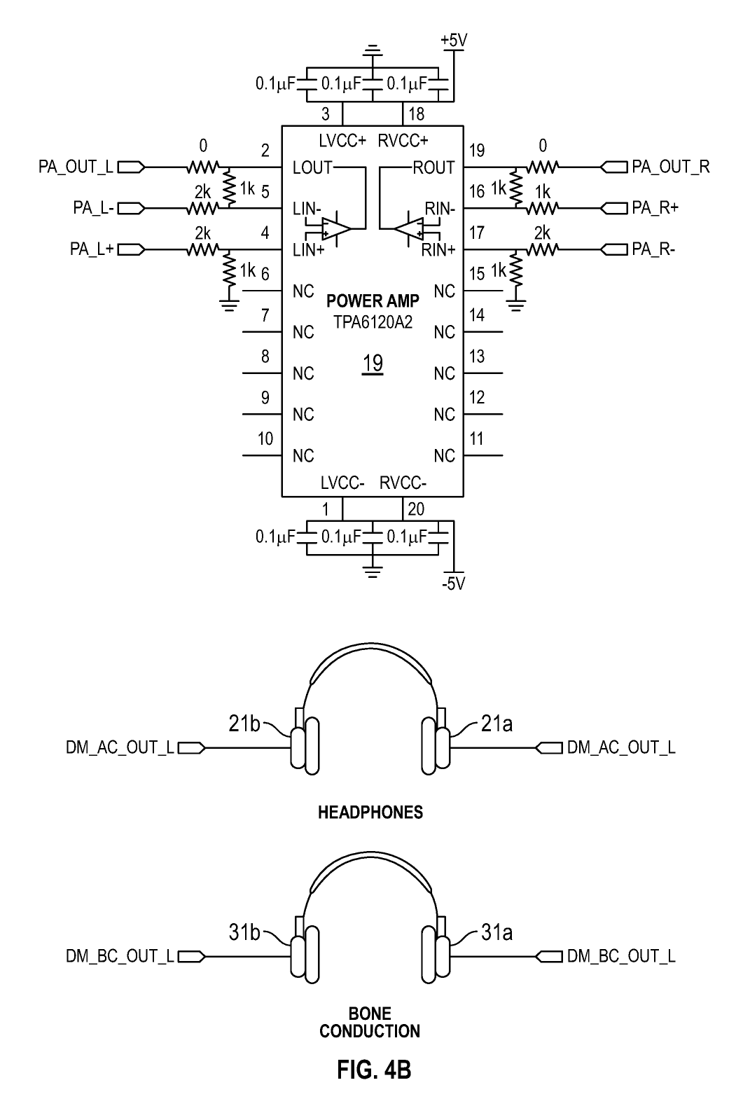

FIGS. 4A-4B are a second part of the circuit schematic (the first part being depicted in FIGS. 3A-3D). FIGS. 4A-4B show the component level integrated circuit 15 that implements the left and right channel pre-amplifiers 15a, 15b. It also shows the component level integrated circuit 19 that implements the left and right output amplifiers 19a and 19b.

The output transducers 21a, 21b shown in the block diagram of FIG. 2 comprise loudspeakers. However, they could instead comprise bone conduction transducers 31a, 31b as shown in FIGS. 4A-4B. In that case a demux chip 33 is provided to switch the output signal from the power amplifier 19 between the loudspeaker and bone conduction transducers as desired. The demux chip 33 is controlled by the FPGA via the DEMUX interface 32 on that chip.

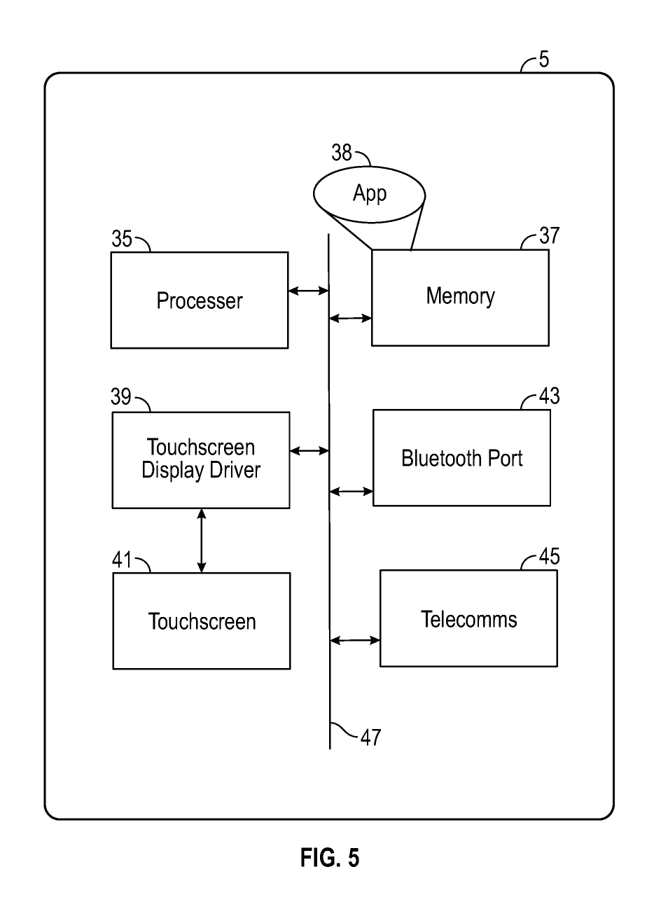

Turning now to FIG. 5 there is depicted a block diagram of the smartphone 5. The smartphone 5 comprises a number of modules which are able to exchange data and commands via a data bus 47. The various modules comprise:

a processor 35;

an electronic memory 37;

a communications module in the form of Bluetooth port 43 for communicating with corresponding Bluetooth port 9 of the transducer portion 7;

a telecoms module 45 which allows the smartphone 5 to establish voice and data communications with a telecommunications network; and

a touchscreen drive module 39, which drives touchscreen 41 and processes user data inputs received via the touchscreen and passes them to the processor 35.

It will be realised that the architecture shown in FIG. 5 is highly simplified and omits many components that are found in a smartphone. However it will be sufficient for those skilled in the art to understand the preferred embodiment of the invention.

The memory 37 stores instructions that comprise a custom application, i.e. “App” 38 which the processor 35 executes in use in order to perform a method according to a preferred embodiment of an aspect of the present invention which will now be described. The programming of the App 38 is straightforward once the method, which will become apparent from the following discussion, is understood.

Referring again to FIG. 1, in use the user 3 dons the headphones 7 and switches the headphones and the smartphone 5 on so that Bluetooth communication is established there between.

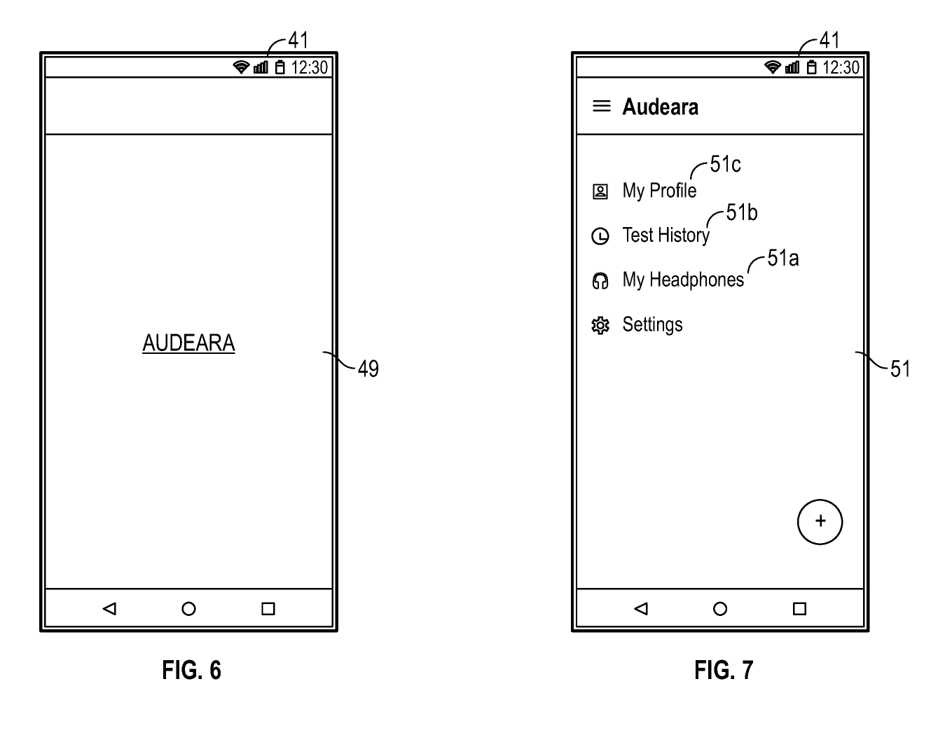

The user then operates the smartphone to initiate execution of the App 38, for example by clicking on an icon for the App, which displayed on touch screen 41. A splash screen 49 shown in FIG. 6 is then displayed to the user 3 and shortly thereafter a control menu screen 51 is displayed as shown in FIG. 7.

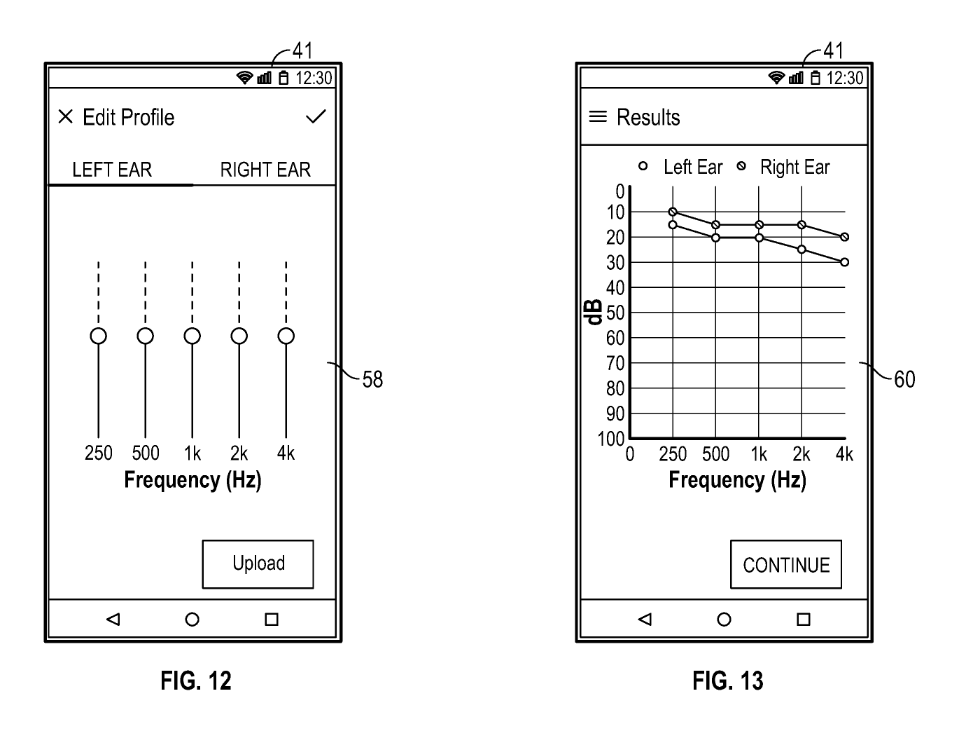

The control menu screen 51 presents the user 3 with three configuration options, 51a, 51b, 51c. The first option is “My Headphones” 51a. If the user has never used the App before and wants to quickly upload some equaliser style adjustments then he/she can choose the “My Headphones” option 51a. in response to that selection the processor 35 presents an equaliser screen 59 shown in FIG. 12. The App is programmed so that the user 3 can quickly set and upload equaliser preferences.

Alternatively if the user 3 has used the app 38 before and has pre saved hearing profiles then the user can choose a second option being “Test History” option 51b. In response to selecting the “Test History” option 51b the processor causes the display of a list view of previous models from which the user can select from and upload to the headphones 7 with or without an equaliser overlay.

Finally, if the user 3 is using the app 38 for the first time then the user can select the “My Profile” option 51c. Selecting the “My Profile” option 51c causes the processor to call up an audio modeling routine and set a personalised model to be uploaded with or without an equaliser overlay to the headphones 7. If an equalizer overlay is applied then the gain adjustment weights that have been determined based on the audiological testing are varied to take into account the user's equalization preferences. For example if the user prefers a bassier sound then the weights corresponding to lower frequency bands are increased.

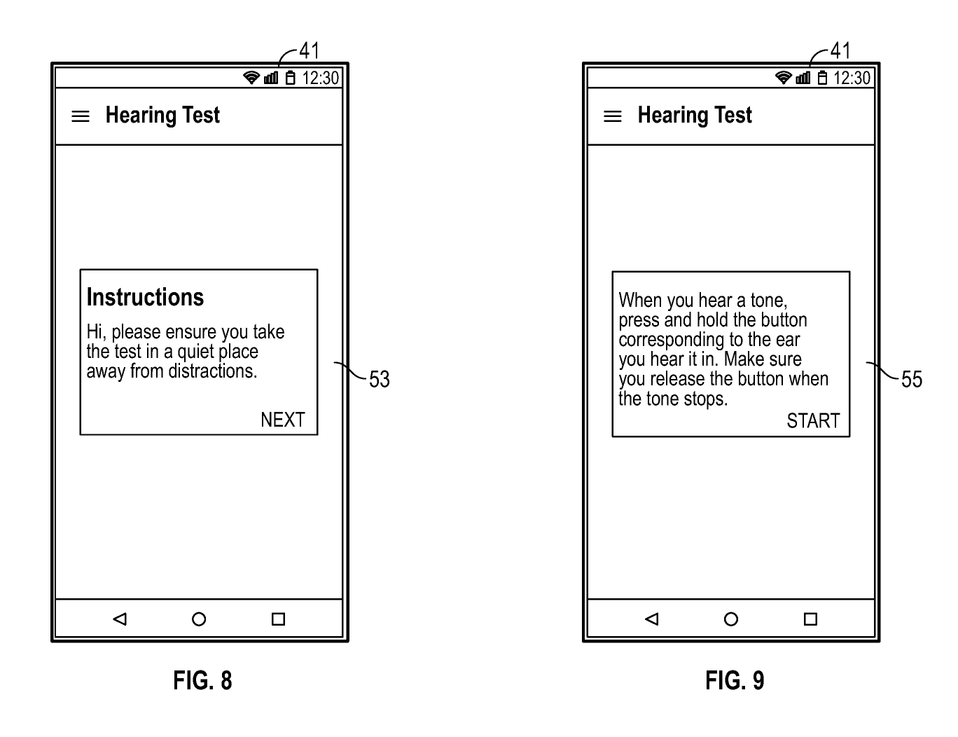

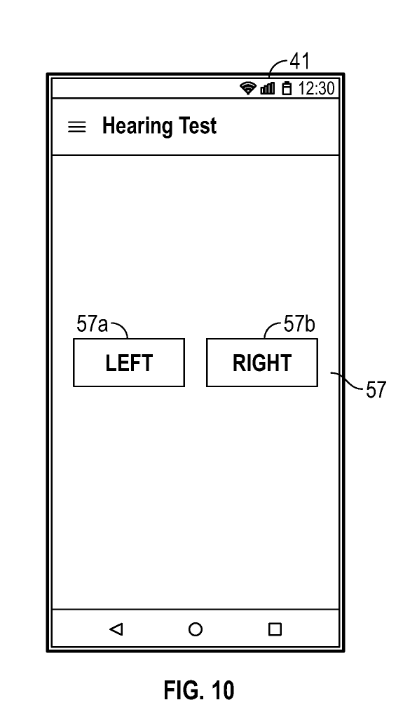

On selecting the “My Profile” option 51c the processor 43 causes screens to display prompting the user to help optimise the acoustic model as displayed in prompt screens 53 (FIG. 8) and 55 (FIG. 9).

Once the user 3 operates touch screen 41 to indicate that he/she is ready to undergo the audio model routine the app 43 displays the interface screen 57 and the user 43 is directed to respond to the software by pressing the “left” 57a or “right” 57b buttons. Upon doing so the processor communicates with the headphones 7 via the Bluetooth link to cause the loudspeakers in the headsets to present beeps in the user's left or right ear respectively.

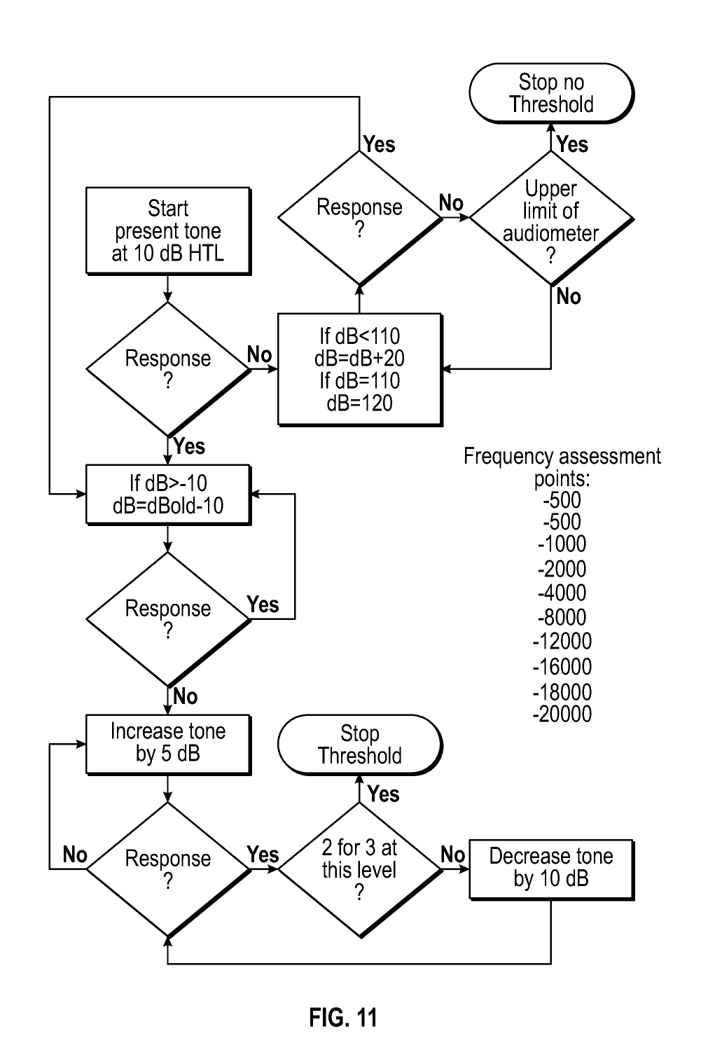

The App then presents screens to step the user through a modeling method 59 that is shown in FIG. 11 and which is in accordance with a preferred embodiment of the invention. The modeling method 59 includes steps to identify the user's minimal perceived headset specific decibel threshold at each of a number of frequency assessment points. The determined specific decibel thresholds for the user are then saved into the audio model. The db Threshold variable for each frequency is set in the boxes titled “Stop Threshold”, in this step the assessment is stopped and the calculated db value is saved; alternatively in the box “Stop No Threshold” the procedure is stopped and the correction db is set to the maximum as the user has profound hearing loss at this frequency and has maximised the capabilities of the hardware. In the flowchart the box labelled “2 for 3 comprises part of an error check loop. The user will actually be presented with that db level three times before the program exits. If the user can hear it two out of three times then they are deemed to be able to hear it. The purpose of this is to avoid input error and the like. The audio model is a set of parameters for the user, including the decibel thresholds that are saved in the digital memory 37.

On completion of the method 59 for each of the frequency assessment points, the App has successfully modeled the way in which the user perceives sound through the headset 7. The app 3 then converts the perceived model into a graphical depiction 60 as shown in FIG. 13 for review. The graph in FIG. 13 showing shows the user's left ear and right ear hearing response as assessed at each of a number of frequencies.

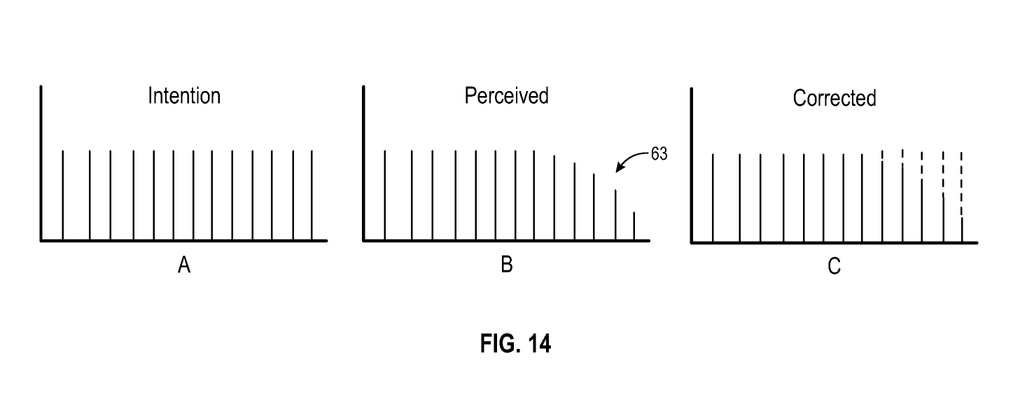

With reference to frequency spectrum graphs of FIG. 14, the method 59 finds the user's perceived gain deficiency 63 in each specific frequency band in the audio spectrum. It then calculates weights (i.e. gain correction factors) by which future audio waveforms need to be gain adjusted in the frequency domain to correct the user's waveform back to perceived unity as intended. These weighting coefficients are then up loaded from the smartphone 5 into the headset FPGA 7. The FPGA then uses the uploaded weights in run time for the dynamic real time processing of uncompensated audio from the smartphone.

The App 43 then displays the equaliser screen 58 (FIG. 12) to the user 3. Here the user has the option to remain with a unity correction as per the audiological model or to use this base level correction with an equaliser overlay to allow the additional personalisation.

Once the user completes this customisation and selects “Upload”, the model with or without the equaliser overlay as per the users preference are transitioned into frequency based coefficient corrections and are uploaded to the paired headset for configuration of the on-board signal processing corrections.

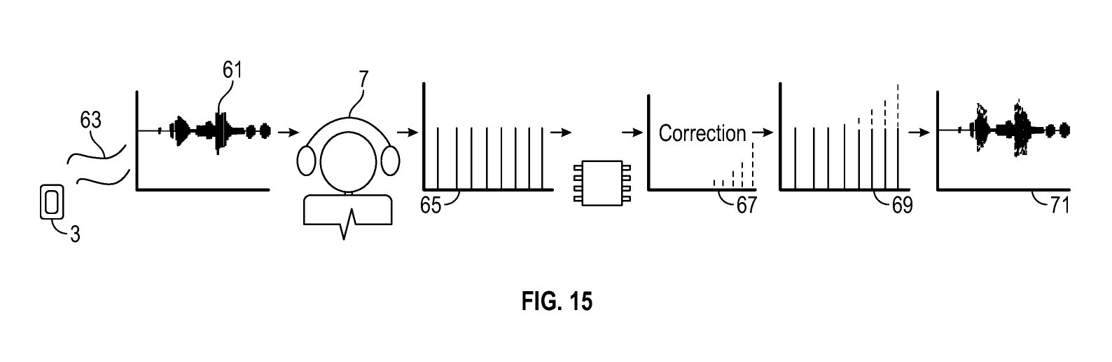

FIG. 15 is a flowchart showing how the FPGA is programmed to use the determined gain adjustment weights, i.e. the audiological model, to process a WAV file (or other audio file) to thereby apply the weightings produced in the audiological assessment with or without an equalizer overlay.

From left to right of FIG. 15, a digital audio signal 61 is transmitted to the headset 7 via a Bluetooth link 63. This received signal is transmitted into the on board FPGA (item 11 of FIG. 2) for signal processing. Inside the FPGA the time domain audio signal 61 is converted to the frequency domain 65 by the utilisation of an FFT this frequency domain signal is then gain adjusted against the patient's personal correction weightings 67 for each corresponding frequency bin to create a gain adjusted frequency domain representation of the signal. The FPGA 11 then undertakes an IFFT to render the signal in to a user specific time domain digital audio waveform 71. The digital audio waveform is then processed by the DAC (item 13 of FIG. 2) and analogue amplifiers and possibly noise cancellation modules to drive the transducers of the headphones.

It will therefore be understood that in a preferred embodiment of the invention a sound delivery system 1 (FIG. 1) is provided. The sound delivery system 1 includes at least one processing assembly which in the presently described embodiment includes the smartphone processor 35 and the headphone FPGA 11. A user interface is provided in the form of smartphone touchscreen 41 and touchscreen display driver unit 39. The touchscreen display driver unit 39 is coupled to the processor via a data bus 47. The sound delivery system 1 also includes at least one audio transducer. For example, either or both loudspeakers 21a, 21b (FIG. 4) and bone conduction transducers 31a, 31b (FIG. 4) are provided. The bone transducers are responsive to signals from the FPGA via suitable digital to analog converts and analog amplifiers.

The at least one processing assembly includes the processor 35 of smartphone 5. That processor is arranged, by virtue of it executing the instructions comprising app 38 that are stored in digital memory 37, to determine compensatory weights at each of a number of audio frequencies for the user. The processor determines the weights on the basis of user responses via the interface (e.g. touchscreen 41) to sounds delivered via the audio transducer (for example the loudspeakers of headset 7). The at least one processor also includes the FPGA 11 which is configured with the determined weights and which is therefore able to deliver audio signals to the user by modifying the audio signals in accordance with the determined weights.

In the presently described embodiment of the invention the user interface portion and the transducer portion of the sound delivery system are physically separate, though in data communication via a Bluetooth connection. It will be realized that in other embodiments of the invention the separation of the two units may not be so. For example, the headset could have a user interface, for example one or more buttons, mounted to the side which are coupled to an internal processor so that a user may initiate the automatic audiological assessment and then press one or other of the buttons to indicate a hearing threshold for a presented audio signal. Such an arrangement would not require a separation of the user interface portion and the transducer (i.e. headset) portion. In such an embodiment the processing assembly might comprise a single, suitably programmed, high frequency processor that is capable of both running the audiological assessment method and also performing the FFT and IFFT functions with gain adjustment according to the determined weights for the user.

In compliance with the statute, the invention has been described in language more or less specific to structural or methodical features. The term “comprises” and its variations, such as “comprising” and “comprised of” is used throughout in an inclusive sense and not to the exclusion of any additional features. It is to be understood that the invention is not limited to specific features shown or described since the means herein described comprises preferred forms of putting the invention into effect. The invention is, therefore, claimed in any of its forms or modifications within the proper scope of the appended claims appropriately interpreted by those skilled in the art.

Throughout the specification and claims (if present), unless the context requires otherwise, the term “substantially” or “about” will be understood to not be limited to the value for the range qualified by the terms.

Any embodiment of the invention is meant to be illustrative only and is not meant to be limiting to the invention. Therefore, it should be appreciated that various other changes and modifications can be made to any embodiment described without departing from the spirit and scope of the invention.

We claim:

1. A sound delivery system comprising:
at least one processing assembly;
an interface for a user coupled to the at least one processing assembly; and
at least one audio transducer responsive to the at least one processing assembly for delivering sound to the user;
wherein the at least one processing assembly is arranged to determine compensatory weights at each of a number of audio frequencies for the user on the basis of user responses via the interface to sounds delivered via the audio transducer and to deliver audio signals to the user modified in accordance with the determined weights via said audio transducer.

2. The system according to claim 1, further comprising:
an interface portion which includes the user interface and a transducer portion which includes the at least one audio transducer,
wherein the first interface portion and the transducer portion include corresponding data communication assemblies for data communication therebetween.

3. The system according to claim 2, wherein the at least one processing assembly includes:
at least one interface processor that is mounted within the interface portion and coupled to the user interface; and
at least one transducer processor that is mounted within the transducer portion and arranged to process sound signals for delivery as sound by said audio transducer.

4. The system according to claim 4, wherein the interface portion comprises a portable computational device such as a smartphone or tablet and wherein the transducer portion comprises a set of headphones.

5. The system according to claim 2, wherein the corresponding data communication assemblies comprise Bluetooth communication assemblies.

6. The system according to claim 4, wherein the transducer portion includes a non-volatile memory that is arranged to retain the weights subsequent to depowering of the transducer portion.

7. The system according to claim 3, wherein the at least one transducer processor is configured to perform a fast Fourier transform (FFT) of the audio signals and to apply the weights to the audio signals in a frequency domain resulting from the FFT.

8. The system according to claim 4, wherein the portable computational device is programmed to prompt the user to identify a left side or a right side of the set of headphones from which a test audio signal is emanating.

9. The system according to claim 4, wherein the portable computational device is programmed to present sounds to the user at a range of frequencies and to increase or decrease the sounds at each of said frequencies to thereby determine a hearing threshold for the user at each of said frequencies wherein the computational device is programmed to determine the weights taking into account the determined hearing threshold for the user at each of the frequencies.

10. The system according to claim 4 wherein the portable computational device is programmed to present a user equalization preference screen for the user to submit audio equalization preferences and to determine the weights taking into account submitted audio equalization preferences of the user.

11. A sound delivery system comprising:
at least one processing assembly;
an interface coupled to the at least one processing assembly;
at least one audio transducer responsive to the at least one processing assembly for delivering sound to a user; and
an electronic memory accessible by the at least one processing assembly storing:
instructions for the processor to determine compensatory weights at each of a number of audio frequencies for the user on the basis of user responses via the interface to sounds delivered via the audio transducer and to deliver audio signals to the user modified in accordance with the determined weights via said audio transducer.

12. An automatic audiological testing apparatus comprising:
at least one processor;
an electronic memory in communication with the processor and containing instructions for execution thereby; and
a user interface in communication with the processor;
wherein the electronic memory stores instructions for the processor to determine compensatory weights at each of a number of audio frequencies for the user on the basis of user responses via the interface to sounds at a number of different frequencies.

13. The apparatus of claim 12 comprising a programmed smartphone.

14. A sound delivery apparatus comprising:
right and left loudspeakers for delivery of sounds to a user:
at least one processor configured to receive gain adjustment weights for a number of different frequency sounds at a number of predetermined frequencies and including a non-volatile memory to store said weights;
wherein the processor is arranged to convert an audio signal into the frequency domain, apply the gain adjustment weights to the audio signal in the frequency domain and convert the adjusted audio signal back into the time domain for delivery to the user via the loudspeakers.

15. A method for sound delivery to a user comprising the steps of:
presenting sounds of different frequencies and prompts to a user by means of a programmed personal computational device such as a smartphone or tablet in order to determine an audiological model of the user comprising a set of gain adjustment weights for each of the different frequencies;
transferring the determined weights to a headset containing transducers for delivery of audio signals to the user; and
adjusting audio signals according to the adjustment weights with a processor of the headset to thereby deliver adjusted audio signals to the user to compensate for hearing deficiencies of the user;
wherein the adjustment weights are stored in a non-volatile memory of the headset.

16. The method of claim 15, further comprising the step of operating the personal computational device to facilitate adjustment of the weights by the user to introduce frequency equalization parameters selected by the user for each of a number of frequency bands.
